(19) United States
(12) Patent Application Publication
(10) Pub. No.: US 2010/0217340 A1
(43) Pub. Date: Aug. 26, 2010

(54) Implantable Medical Device Connector System

(75) Inventors: Brian P. Watschke, Eden Prairie, MN (US); John Jason Buysman, Minnetonka, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/710,923

(22) Filed: Feb. 23, 2010

Related U.S. Application Data
(60) Provisional application No. 61/154,483, filed on Feb. 23, 2009.

(51) Int. Cl. A61N 1/375; A61N 1/05

(52) U.S. Cl. 607/2; 607/116

(57) ABSTRACT

An implantable medical stimulation device is provided including non-weld connections between one or more feed-through conductors and electrical contacts of a connector block for the device. The device can be configured for implantation into a pelvic region of a patient to provide muscle and/or nerve stimulation that is used to control and/or treat a pelvic condition of the patient, such as pelvic pain, urinary incontinence, fecal incontinence, erectile dysfunction or other pelvic conditions. The non-weld connections serve to simplify connectivity by providing an insertable wedge-like member, or a crimping member, adapted to facilitate selective electrical connectivity.

### RELATED APPLICATION

This application claims priority to and the benefit of U.S. Provisional Application No. 61/154,483, filed Feb. 23, 2009, which is incorporated herein by reference in its entirety.

### FIELD OF THE INVENTION

The present invention relates generally to medical electronic devices, and specifically to implantable electrical stimulation devices adapted to treat various pelvic disorders.

### BACKGROUND OF THE INVENTION

Implantable electronic stimulator devices, such as neuromuscular stimulation devices, have been disclosed for use in the treatment of various pelvic conditions, such as urinary incontinence, fecal incontinence and sexual dysfunction. Such devices generally include one or more electrodes that are coupled to a control unit by electrode leads. Electrical signals are applied to the desired pelvic tissue of the patient through the electrode leads in order to treat the condition of the patient. Exemplary implantable electronic stimulator devices and uses of the devices are disclosed in U.S. Pat. Nos. 7,613,516, 7,582,053, 7,387,603 6,354,991, 6,652,449, 6,712,772 6,862,480, U.S. Patent Publication Nos. 2009/0254145, 2009/0012592, and 2009/0043356, each of which is hereby incorporated by reference in its entirety.

In general, certain of these implantable medical devices generally comprise electronics contained within a housing. The electronics can comprise a battery powered pulse generator and/or a microprocessor-based controller.

The device may also facilitate electrical connections between the conductors that are external to the housing, e.g., an implantable conductor, and the electronics within the housing. The housing generally receives a conductor that is electrically coupled to the electronics and extends outside of the housing.

However, conventional techniques and systems require welding the electrical contacts of the connectors. Such a welding process increases the complexity of the manufacturing process and is susceptible to manufacturing error.

### SUMMARY OF THE INVENTION

Embodiments of the invention include systems and methods for treating a pelvic disorder of a patient, such as urinary incontinence fecal incontinence, constipation and pathological retention of urine, for example. In accordance with embodiments of the present invention, at least one electrode of a lead is implanted in contact with a pelvic muscle of the patient. A proximal end of the lead is connected to a hermetically sealed implantable stimulator device configured to apply an electrical waveform through the at least one electrode. The electrical waveform is delivered from the stimulator device to the pelvic muscle through the lead and the at least one electrode. The electrical waveform can provide at least partial relief from urinary incontinence, fecal incontinence, constipation or pathological urine retention.

In various embodiments, the implantable stimulator device comprises non-weld connections between one or more feed-through conductors and electrical contacts of a connector block for the device to simplify connectivity and reduce manufacturing time and costs. For instance, one embodiment can include an insertable wedge-like member adapted to facilitate electrical connectivity. Other embodiments can include a crimping member adapted to facilitate electrical connectivity.

### BRIEF DESCRIPTION OF THE DRAWINGS

FIG. 1A is a partial schematic view of an implantable medical stimulator device in accordance with embodiments of the present invention.

FIG. 1B is a partial schematic view of an implantable medical stimulator device depicting various electrical connections in accordance with embodiments of the present invention.

FIGS. 2A-C are partial schematic views of an implantable medical stimulator device illustrating electrical coupling via a wedge member of a feed-through conductor to electrical contacts of a connector block, in accordance with embodiments of the invention.

FIGS. 3A-E are partial schematic views of an implantable medical stimulator device illustrating electrical coupling via a crimping member of a feed-through conductor to electrical contacts of a connector block, in accordance with embodiments of the invention.

### DETAILED DESCRIPTION OF THE EMBODIMENTS

Embodiments of the present invention are generally directed to an implantable medical device 100 comprising non-weld connections between one or more feed-through conductors and electrical contacts of a connector block for the device. Device 100 is configured for implantation into a pelvic region of a patient to provide muscle and/or nerve stimulation that is used to control and/or treat a pelvic condition of the patient, such as pelvic pain, urinary incontinence, fecal incontinence, erectile dysfunction or other pelvic condition that may be treated through electrical stimulation. Exemplary implantable electronic stimulator devices, systems and methods and methods are disclosed in U.S. Pat. Nos. 7,613,516, 7,582,053, 7,387,603 6,354,991, 6,652,449, 6,712,772 6,862,480, U.S. Patent Publication Nos. 2009/0254145, 2009/0012592, and 2009/0043356, each of which is hereby incorporated by reference in its entirety. The various electronic stimulator devices, systems and methods disclosed in these incorporated references can be implemented, all or in part, with the device 100 of the present invention.

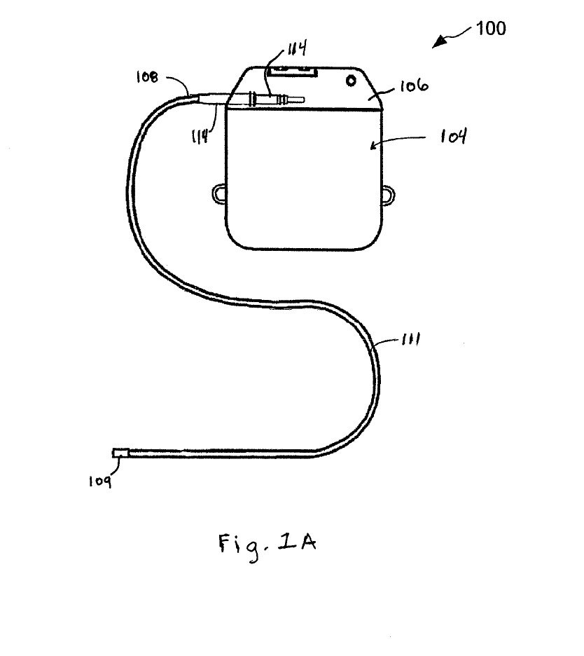

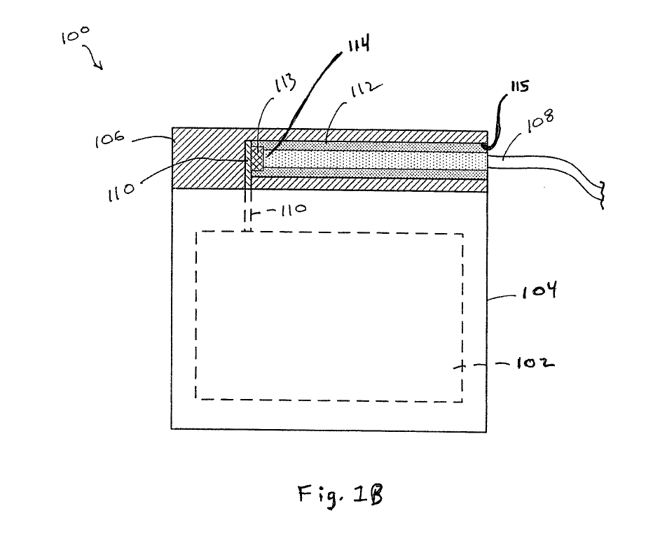

FIG. 1B is a partial schematic diagram of an implantable medical device 100 capable of use with or modification for the present invention. The implantable medical device 100 generally comprises electronics or circuitry 102 contained within a hermetically sealed metal or plastic compatible housing or control unit 104, which is commonly referred to as the “can.” The electronics 102 can comprise any desired electronics that are capable of performing the desired function of the device 100 once implanted in the patient. In one embodiment, the electronics 102 include a battery powered pulse generator and one or more microprocessor-based controllers, in accordance with conventional implantable stimulator devices. The control unit 104 can include first and second halves that are joined together in a laser-welding or similar operation about their perimeters after the electronics 102 are inserted in the space defined by the two halves of the control unit 104.

The device 100 may also include a header 106, which attaches to the control unit 104 and facilitates electrical connections between the conductors that are external to the control unit 104, e.g., an implantable conductor 108 such as electrode leads or a lead wire, to the electronics 102 within the control unit 104. Each electrode lead 108 can comprise a lead body 111 and one or more stimulating electrodes 109 at a distal end of the electrode lead 108 or lead body. The lead body 111 insulates electrical wires connecting the device control unit 104 to the stimulating electrodes 109. The lead body 111 can be in the form of an insulating jacket typically comprising silicone, polyurethane or other flexible, biocompatible electrically insulating materials. Additional electrode leads 108 or physiological sensors may be coupled to the device 100, or a portion thereof. Further, the leads or electrodes can be coupled or otherwise provided with various mesh devices, slings, and like devices or systems adapted to treat various pelvic disorders.

In one embodiment, the electronics 102 include circuitry for processing electrical signals received from the one or more stimulating electrodes 109 or physiological sensors. The electronics 102 can also be configured to apply an electrical current or waveform to the tissue of the patient that is in contact with the one or more stimulating electrodes 109. The electrode lead 108 and/or electrode 109 can be anchored to pelvic tissue of the patient (e.g., internal urinary sphincter muscle) by means of a tissue anchor or anchoring systems as disclosed in the previously incorporated patent references.

The device 100 can be employed to treat urge incontinence. The electrode or electrodes can be implanted in the pelvic region of a patient so as to contact one or more of the muscles or nerves that are used in regulating urine flow from the bladder. The control unit is preferably implanted under the skin of the abdomen or genital region, and receives signals from the electrodes and/or from the sensors. When the control unit determines that the signals are indicative of impending involuntary urine flow from the bladder, it can apply a suitable electrical waveform to the electrode or electrodes, stimulating the contacted muscle or nerve to inhibit the urine flow. The present invention can be employed in various pelvic treatment scenarios, such as those known or disclosed in the previously-incorporated references.

The header 106 generally receives a feed-through conductor 110 that is electrically coupled to the electronics 102 and extends outside of the control unit 104 through or to the lead 108. The header 106 can include a connector block 112 that may be molded in the header 106, inserted after the header 106 has been formed, or otherwise provided with the header 106. The feed-through conductors 110 are coupled to one or more electrical contacts 113 of the connector block 112. The connector block 112 can include one or more ports 115, each of which receives a proximal end 114 of the implantable conductor (e.g., lead) 108 and electrically operably couples the conductor 108 to the electronic circuitry 102 of the control unit 104 via the electrical contacts 113 and the feed-through conductors 110.

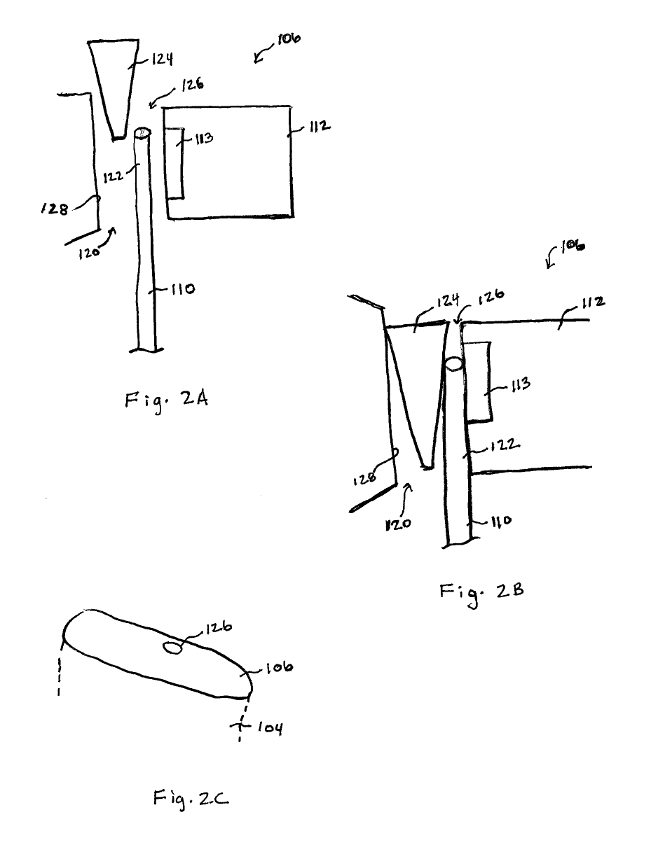

FIGS. 2A-C illustrate a device 100 and a method of operably electrically coupling a feed-through conductor 110 to electrical contacts of the connector block 112, in accordance with embodiments of the invention. In one embodiment, the header 106 includes an opening 120 that receives an end 122 of the feed-through conductor 110. A wedge or member 124 can be inserted through an access port 126 in the header 106 and driven into the access port 126 such that a portion of the wedge engages an interior wall 128 of the opening 120 and drives the end 122 of the conductor 110 against the electrical contacts 113 of the connector block 112, as shown in FIG. 2B. Alternatively, when the wedge 124 is electrically conductive, the wedge 124 can be placed between the end 122 of the conductor 110 and the electrical contacts 113 of the connector block 112 to provide the desired electrical connection or communication between the electrical contacts 113 and the electronics 102.

In one embodiment, the wedge 124 is a rigid member that is suitable for implantation in a patient. In another embodiment, the wedge 124 is semi-rigid or semi-flexible and can deform slightly in response to the pressure between the interior wall 128 and the conductor 110. Further, the wedge 124 can instead take on various shapes and configurations of a straight, arcuate or similar member not having distinct tapering.

The access port 126, through which the wedge 124 is inserted in the header 106, can be located as desired on the header 106 such that it provides access to the opening 120 where the end 122 of the conductor 110 is located. Thus, the access port 126 can be positioned on a top side of the header 106 that is opposite the control unit 104, on a side of the header 106, or elsewhere according to varying configurations and needs. The access port 126 can be sealed using conventional techniques to secure the wedge 124 in place and prevent fluids from entering the opening 120 during use.

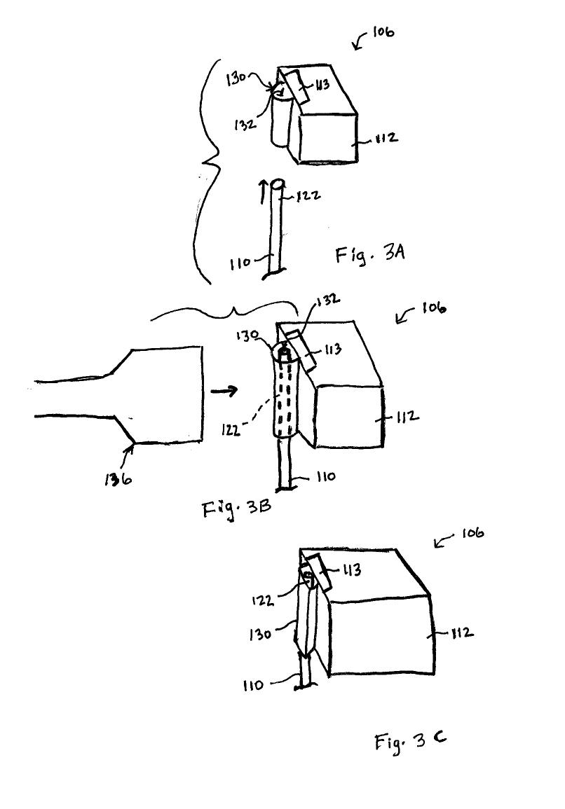

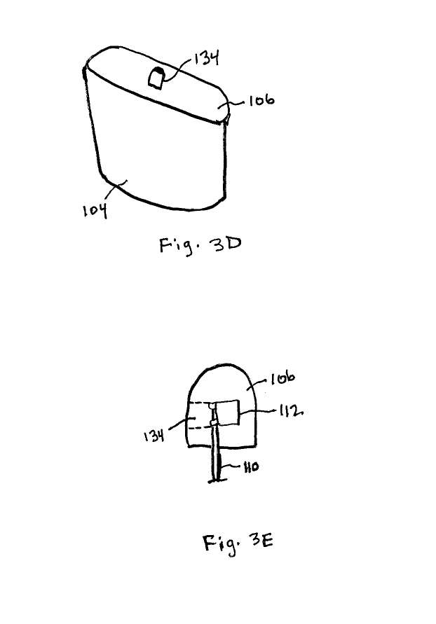

FIGS. 3A-E illustrate a manner of operably electrically coupling one or more feed-through conductors 110 to the electrical contacts 113 of the connector block 112, in accordance with embodiments of the invention. In one embodiment, the header 106 comprises a deformable crimping member 130 (e.g., tube) that defines an opening 132 adjacent the electrical contacts 113 of the connector block 112. The crimping member 130 is a deformable member that, once deformed, generally maintains the deformed position. The opening 132 is configured to receive the end 122 of the conductor 110, as illustrated in FIG. 3B. The crimping member 130 is deformable to secure the end 122 of the conductor 110 against the electrical contacts 113 to provide the desired electrical connection and communication between the electrical contacts 113 and the electronics 102 without welding.

In one embodiment, the header 106 includes an access port 134 (e.g., FIGS. 3D-3E) through which a crimping tool 136 (FIG. 3B) can be inserted to deform the crimping member 132 such that it presses the end 122 of the conductor 110 against the electrical contacts 113 of the connector block 112, as shown in FIG. 3C. The access port 134 can be positioned anywhere along the header 106, e.g., along a side of the header 106 as shown in FIG. 3E, which allows the crimping tool 136 to be inserted through the access and pressed against the crimping member 130 to deform the crimping member 130 and press the conductor 110 against the electrical contacts 113 of the connector block 112.

In accordance with another embodiment, the crimping tool 136 can include a wedge, such as that illustrated in FIGS. 2A and 2B, which deforms the crimping member 130 and drives the end 122 of the conductor 110 against the electrical contacts 113 of the connector block 112 responsive to its insertion in the access port 134.

In certain embodiments, the crimping member 130 can be electrically conductive. Further, the crimping member 130 can form one of the electrical contacts 113 for the connector block 112.

The access port 134 can be sealed using conventional techniques to prevent fluids from reaching the crimping member 130, the conductor 110 and the electrical contacts 113. In addition, various caps, seals or other devices, techniques or methods can be employed with various embodiments of the present invention to close off the various ports, openings or exposed areas of the device 100 to prevent fluids from reaching certain parts or components of the device 100.

The devices, systems and their various components, structures, features, materials and methods may have a number of suitable configurations as shown and described in the previously-incorporated references. All patents, patent applications, and publications cited herein are hereby incorporated by reference in their entirety as if individually incorporated, and include those references incorporated within the identified patents, patent applications and publications.

A variety of materials may be used to form portions, structures or components of the devices and systems described herein, including nitinol, polymers, elastomers, thermoplastic elastomers, metals, ceramics, springs, wires, plastic tubing, and the like.

Obviously, numerous modifications and variations of the present invention are possible in light of the teachings herein. It is therefore to be understood that within the scope of the appended claims, the invention may be practiced other than as specifically described herein.

What is claimed is:

1. An implantable stimulator device, comprising:
a control unit including;
electronic circuitry;
a feed-through conductor operably coupleable to the electronic circuitry and extendable outside of the control unit;
a header including a connector block having an electrical contact, and an opening adjacent the electrical contact configured to receive an end of the feed-through conductor;
an access port to the opening adjacent the electrical contact; and
a member insertable through the access port, between an interior wall of the opening and the end of the feed-through conductor, to secure the conductor in electrical communication with the electrical contact.

2. The device of claim 1, wherein the member is generally tapered to define a wedge member.

3. The device of claim 1, wherein at least one of the member or the end of the feed-through conductor is generally deformable.

4. The device of claim 1, wherein the member is electrically conductive.

5. The device of claim 1, wherein a distal end of the feed-through conductor includes an electrode adapted for stimulating tissue.

6. The device of claim 1, further including means for sealing off at least a portion of the access port to prevent fluid from entering the access port.

7. The device of claim 1, wherein the feed-through conductor further includes a lead body extending outside of the control unit.

8. The device of claim 1, wherein the control unit is adapted for implantation under the skin of the abdomen or genital region of a patient.

9. An implantable stimulator device, comprising:
a device housing including electronics;
a feed-through conductor operably coupleable to the electronics and extendable outside of the device housing; and
a header including a connector block having an electrical contact, and an opening adjacent the electrical contact configured to receive an end of the feed-through conductor; and
a crimping member adjacent the electrical contact and adapted to secure the end of the conductor in electrical communication with the electrical contact.

10. The device of claim 9, further including a crimping tool adapted to crimp the crimping member.

11. The device of claim 9, wherein the crimping member is a deformable tube member.

12. The device of claim 9, wherein the crimping member is electrically conductive.

13. The device of claim 9, wherein a distal end of the feed-through conductor includes an electrode adapted for stimulating tissue.

14. The device of claim 9, further including means for sealing off at least a portion of the opening to prevent fluid from entering the opening.

15. The device of claim 9, wherein the feed-through conductor further includes a lead body extending outside of the control unit.

16. The device of claim 9, wherein the device housing is adapted for implantation under the skin of the abdomen or genital region of a patient.

17. A method of providing electrical stimulation to a patient, comprising:
providing an implantable stimulator device adapted for implantation in a patient, the implantable stimulator device including a control unit and a header, the control unit housing electronics, and the header including a connector block having an electrical contact and an opening adjacent the electrical contact, with a feed-through conductor operably and electrically coupleable to the electronics;
inserting an end of the feed-through conductor into the opening;
manipulating a member to provide electrical communication between the feed-through conductor and the electrical contact; and
implanting the stimulator device in the pelvic region of the patient.

18. The method of claim 17, wherein the member is a tapered wedge member.

19. The method of claim 17, wherein the member is a crimping member.

20. The method of claim 17, wherein the member is electrically conductive.
